(19) United States
(12) Patent Application Publication
(10) Pub. No.: US 2009/0131793 A1
(43) Pub. Date: May 21, 2009

(54) PORTABLE IMAGING SYSTEM HAVING A SINGLE SCREEN TOUCH PANEL

(75) Inventors: Andrew David Stonefield, Whitefish Bay, WI (US); Menachem Halmann, Milwaukee, WI (US); Giorgio Grimaldi, Meudon (FR)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/940,491

(22) Filed: Nov. 15, 2007

(51) Int. Cl. A61B 8/13

(52) U.S. Cl. 600/443

(57) ABSTRACT

A portable imaging system is presented. The system includes a single panel display, where the single panel display includes a first portion configured to display an image and a second portion configured as a touch-based user interface. A method of imaging using a portable imaging system, where the portable imaging system includes a single panel display, where the single panel display includes a first portion configured to display an image and a second portion configured as a touch-based user interface, a controls portion, where the controls portion includes one or more buttons configured to aid in performing commonly used functions, is also presented. The method includes displaying an image on the first portion of the single panel display. In addition, the method includes manipulating the image using controls on the touch-based user interface.

### BACKGROUND

This disclosure relates generally to diagnostic imaging methods and apparatus, and more particularly, to a user interface for the diagnostic imaging apparatus.

Diagnostic imaging has emerged into an essential aspect of patient care. Medical images that are obtained during a diagnostic imaging session have evolved as tools that allow a clinician non-invasive means to view anatomical cross-sections of internal organs, tissues, bones and other anatomical regions of a patient. More particularly, the medical images serve the clinician in diagnosis of disease states, determination of suitable treatment options and/or monitoring the effects of treatment, to name a few. As will be appreciated, medical images may be obtained from a broad spectrum of imaging modalities, such as, but not limited to computed tomography (CT) imaging, ultrasound imaging, magnetic resonance (MR) imaging, digital mammography, X-ray imaging, nuclear medicine imaging, or positron emission tomography (PET) imaging.

Ultrasound imaging (also referred to as ultrasound scanning or sonography) is a relatively inexpensive and radiation-free imaging modality. As will be appreciated, ultrasound typically involves non-invasive imaging and is being increasingly used in the diagnosis of a number of organs and conditions, without X-ray radiation. Further, modern obstetric medicine for guiding pregnancy and childbirth is known to rely heavily on ultrasound to provide detailed images of the fetus and the uterus. In addition, ultrasound is also extensively used for evaluating the kidneys, liver, pancreas, heart, and blood vessels of the neck and abdomen. More recently, ultrasound imaging and ultrasound angiography are finding a greater role in the detection, diagnosis and treatment of heart disease, heart attack, acute stroke and vascular disease which may lead to stroke. Also, ultrasound is also being used more and more to image the breasts and to guide biopsy of breast cancer.

Further, diagnostic imaging systems, such as ultrasound imaging systems typically entail use of a user interface to control scanning operation and a display screen to view images being scanned. Typically, these imaging systems include a separate console and display screen. However, as will be appreciated, some imaging systems may include a box or tablet shaped scanner, with buttons disposed adjacent to the display screen. In either embodiment, the display and the console are generally physically separate components that may be joined together to form the imaging system.

In the case of an ultrasound imaging system, a display screen is used to view images produced by an image acquisition device, such as a probe. Recently, the ultrasound imaging system has been known to include a screen that is often a flat panel framed in plastic without any other protection against chemicals or fluid splatter. Furthermore, in the imaging systems using multiple components there are part lines or seams where the components are joined together, further increasing the risk of contamination by infectious diseases and/or bacteria in a medical environment in which a diagnostic imaging system may be employed. A similar risk of contamination is posed around keypads, mechanical buttons, trackballs, and touch pads that are part of the diagnostic imaging system.

Cleaning the seams between all the components is an onerous task that may have to be performed daily by a clinician in meticulous detail. However, there is a risk that the equipment may not be totally cleaned because small splatters of blood and other bodily fluids may go unseen. To ameliorate this problem, flexible plastic films or sheets that may be layered onto consoles and keyboards have been used. Unfortunately, these drapes or covers tend to interfere with the visibility of images and the operation of the imaging systems and may not always be completely effective in eliminating contamination. In still other cases, imaging systems are placed outside of a sterile field. However, the user then may have to twist and strain just to see an image and an additional person may be required to operate the imaging system.

Additionally, in a sterile environment such as an operating room (OR), it may be desirable to use an imaging system that is relatively small in size, portable, simple to use and easily cleanable. For example, in the OR it may be desirable to use an ultrasound imaging system having a relatively small footprint to visualize non-invasive surgical procedures. Also, if a clinician other than an ultrasonographer is using the ultrasound imaging system, simplicity of the imaging system is important for ease of use. Moreover, working in a sterile field, every crack and seam is a breeding ground of infectious bacteria. Hence, it may be desirable that the ultrasound imaging system be easily cleanable.

It may therefore be desirable to develop a design for a portable imaging system having a relatively small size and simple to use. There also exists a need for an imaging system that may be wipeable and easily cleaned, thus allowing use of the imaging system in sterile environments.

### BRIEF DESCRIPTION

In accordance with aspects of the present technique, a portable imaging system is presented. The system includes a single panel display, where the single panel display includes a first portion configured to display an image and a second portion configured as a touch-based user interface.

In accordance with further aspects of the present technique, a method of making a portable imaging system is presented. The method includes providing a single panel display, where the single panel display includes a first portion configured to display an image and a second portion configured as a touch-based user interface.

In accordance with further aspects of the present technique, a method of imaging using a portable imaging system, where the portable imaging system includes a single panel display, where the single panel display includes a first portion configured to display an image and a second portion configured as a touch-based user interface, a controls portion, where the controls portion includes one or more buttons configured to aid in performing commonly used functions, is presented. The method includes displaying an image on the first portion of the single panel display. In addition, the method includes manipulating the image using controls on the touch-based user interface. Computer-readable medium that afford functionality of the type defined by this method is also contemplated in conjunction with the present technique.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary diagnostic system, in accordance with aspects of the present technique;

FIG. 2 is a block diagram of an exemplary imaging system in the form of an ultrasound imaging system for use in the exemplary diagnostic system of FIG. 1;

FIG. 3 is a diagrammatic illustration of an exemplary portable imaging system, in accordance with aspects of the present technique;

FIG. 4 is a diagrammatic illustration of a side view of the exemplary portable imaging system of FIG. 3, in accordance with aspects of the present technique;

FIG. 5 is a diagrammatic illustration of a method of customizing the portable imaging system of FIG. 3, in accordance with aspects of the present technique;

FIG. 6 is a diagrammatic illustration of a user-defined touch-based user interface in the portable imaging system of FIG. 3, in accordance with aspects of the present technique;

FIG. 7 is a diagrammatic illustration of a method of annotating an image displayed on the portable imaging system of FIG. 3, in accordance with aspects of the present technique;

FIG. 8 is a diagrammatic illustration of one embodiment of a touch-based user interface, in accordance with aspects of the present technique;

FIG. 9 is a diagrammatic illustration of another embodiment of a touch-based user interface, in accordance with aspects of the present technique;

FIG. 10 is a diagrammatic illustration of yet another embodiment of a touch-based user interface, in accordance with aspects of the present technique;

FIG. 11 is a flow chart illustrating a process of making an exemplary portable imaging system, in accordance with aspects of the present technique; and

FIG. 12 is a flow chart illustrating a process of imaging using the exemplary portable imaging system, in accordance with aspects of the present technique.

### DETAILED DESCRIPTION

Although the exemplary embodiments illustrated hereinafter are described in the context of a medical imaging system, it will be appreciated that use of the diagnostic system in industrial applications are also contemplated in conjunction with the present technique. For example, the diagnostic system may find applications in industrial systems such as industrial imaging systems and non-destructive evaluation and inspection systems, such as pipeline inspection systems and liquid reactor inspection systems.

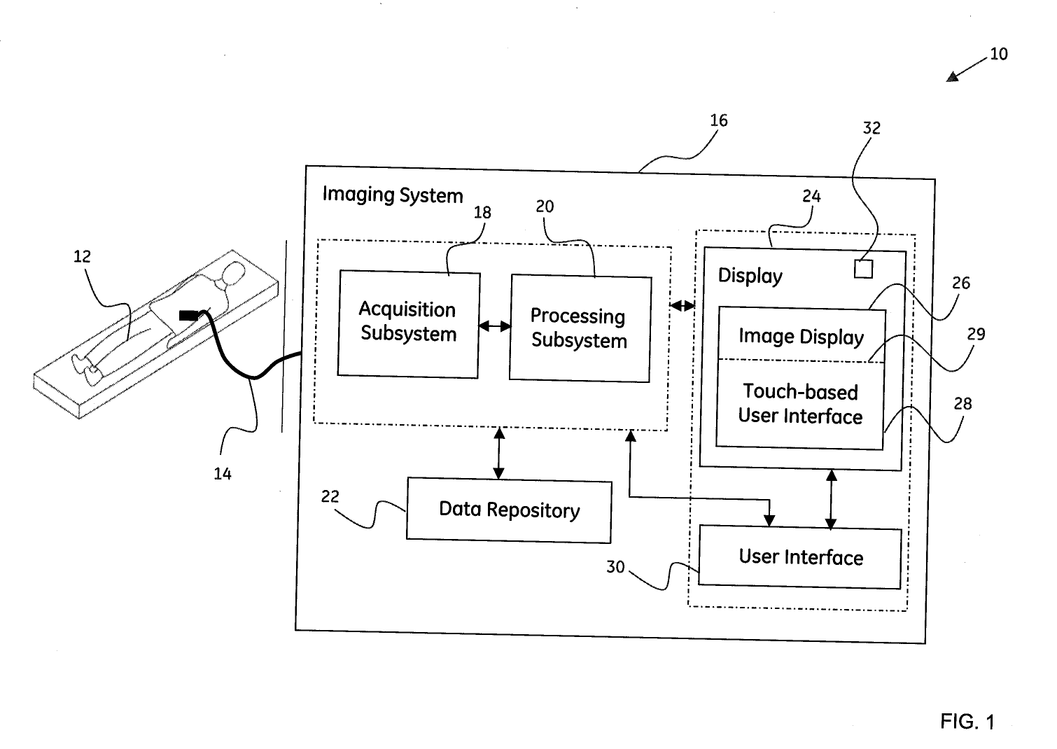

FIG. 1 is a block diagram of an exemplary system 10 for use in diagnostic imaging in accordance with aspects of the present technique. The system 10 may be configured to acquire image data from a patient 12 via an image acquisition device 14. In one embodiment, the image acquisition device 14 may include a probe, where the probe may include an invasive probe, or a non-invasive or external probe, such as an external ultrasound probe, that is configured to aid in the acquisition of image data. By way of example, the image acquisition device 14 may include a probe, where the probe includes an imaging catheter, an endoscope, a laparoscope, a surgical probe, an external probe, or a probe adapted for interventional procedures. The image acquisition device 14 may also include a probe configured to facilitate acquisition of an image volume. It may be noted that the terms probe and image acquisition device may be used interchangeably.

Although the present example illustrates the image acquisition device 14 as being coupled to an imaging system via a probe cable, it will be understood that the probe may be coupled with the imaging system via other means, such as wireless means, for example. Also, in certain other embodiments, image data may be acquired via one or more sensors (not shown) that may be disposed on the patient 12. By way of example, the sensors may include physiological sensors (not shown), such as electrocardiogram (ECG) sensors and/or positional sensors, such as electromagnetic field sensors or inertial sensors. These sensors may be operationally coupled to a data acquisition device, such as an imaging system, via leads (not shown), for example.

The system 10 may also include a medical imaging system 16 that is in operative association with the image acquisition device 14. It should be noted that although the exemplary embodiments illustrated hereinafter are described in the context of a medical imaging system, other imaging systems and applications, such as industrial imaging systems and non-destructive evaluation and inspection systems, such as pipeline inspection systems and liquid reactor inspection systems, are also contemplated. Additionally, the exemplary embodiments illustrated and described hereinafter may find application in multi-modality imaging systems that employ ultrasound imaging in conjunction with other imaging modalities, position-tracking systems or other sensor systems. It may be noted that the other imaging modalities may include medical imaging systems, such as, but not limited to, an ultrasound imaging system, a computed tomography (CT) imaging system, a magnetic resonance (MR) imaging system, a nuclear imaging system, a positron emission topography system or an X-ray imaging system.

In a presently contemplated configuration, the medical imaging system 16 may include an acquisition subsystem 18 and a processing subsystem 20. Further, the acquisition subsystem 18 of the medical imaging system 16 may be configured to acquire image data representative of one or more anatomical regions of interest in the patient 12 via the image acquisition device 14. The image data acquired from the patient 12 may then be processed by the processing subsystem 20.

Additionally, the image data acquired and/or processed by the medical imaging system 16 may be employed to aid a clinician in identifying disease states, assessing need for treatment, determining suitable treatment options, and/or monitoring the effect of treatment on the disease states. In certain embodiments, the processing subsystem 20 may be further coupled to a storage system, such as a data repository 22, where the data repository 22 may be configured to receive and/or store image data.

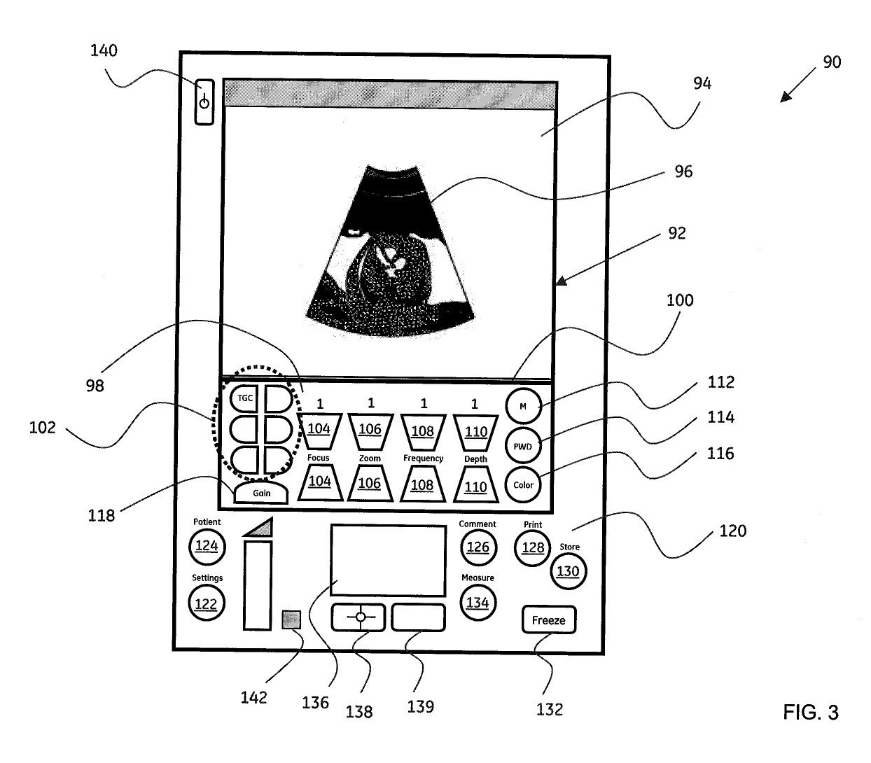

Further, as illustrated in FIG. 1, the medical imaging system 16 may include a display 24 and a user interface 30. However, in certain embodiments, such as in a touch screen, the display 24 and the user interface 30 may overlap. Also, in some embodiments, the display 24 and the user interface 30 may include a common area. In accordance with aspects of the present technique, the display 24 of the medical imaging system 16 may be configured to display one or more images generated by the medical imaging system 16 based on the image data acquired via the image acquisition device 14, and will be described in greater detail with reference to FIGS. 3-12.

In accordance with exemplary aspects of the present technique, the display 24 may be configured to include single panel display. Also, the single panel display 24 may include at least a first portion 26 and a second portion 28. The first portion 26 of the single panel display 24 may be configured to display an image representative of an anatomical region of interest of the patient 12, for example. Additionally, the second portion 28 of the single panel display 24 may be configured for use as a touch-based user interface. The touch-based user interface 28 may be configured to display controls (not shown in FIG. 1), where the controls may be used to perform imaging tasks associated with the imaging system 16. Further reference numeral 29 may be representative of a graphical demarcator configured to virtually divide the single panel display 24 into the first and second portions 26, 28, although use of graphical demarcator 29 is optional and may be omitted in some embodiments. The working of the exemplary single panel display 24, the image area 26 and the touch-based user interface 28 will be described in greater detail with reference to FIGS. 3-12.

In addition, the user interface 30 of the medical imaging system 16 may include a human interface device (not shown) configured to facilitate the clinician in the acquisition of image data representative of the patient 12. The human interface device may include a mouse-type device, a trackball, a joystick, a stylus, or buttons configured to aid the clinician in acquiring image data representative of one or more regions of interest in the patient 12. However, as will be appreciated, other human interface devices, such as, but not limited to, a touch screen, may also be employed. Furthermore, in accordance with aspects of the present technique, the user interface 30 may be configured to aid the clinician in navigating through the images acquired by the medical imaging system 16. Additionally, the user interface 30 may also be configured to aid in manipulating and/or organizing the acquired image data for display on the display 24 and will be described in greater detail with reference to FIGS. 3-12.

According to further aspects of the present technique, the imaging system 16 may also be configured to automatically adjust a brightness of the single panel display 24 based on current ambient conditions. For example, if the ambient condition includes a substantially bright environment, then the imaging system 16 may be configured to enhance the brightness of the single panel display 24. However, if the ambient condition includes a substantially dark environment, then the imaging system 16 may be configured to accordingly dim the brightness of the single panel display 24. In a presently contemplated configuration, the imaging system 16 may be configured to automatically adjust the brightness of the single panel display 24 via use of an ambient light sensor 32. It may be noted that although the ambient light sensor 32 is disposed in the display area 24 in the embodiment illustrated in FIG. 1, it may be appreciated that the ambient light sensor 32 may be disposed at other locations on the imaging system 16.

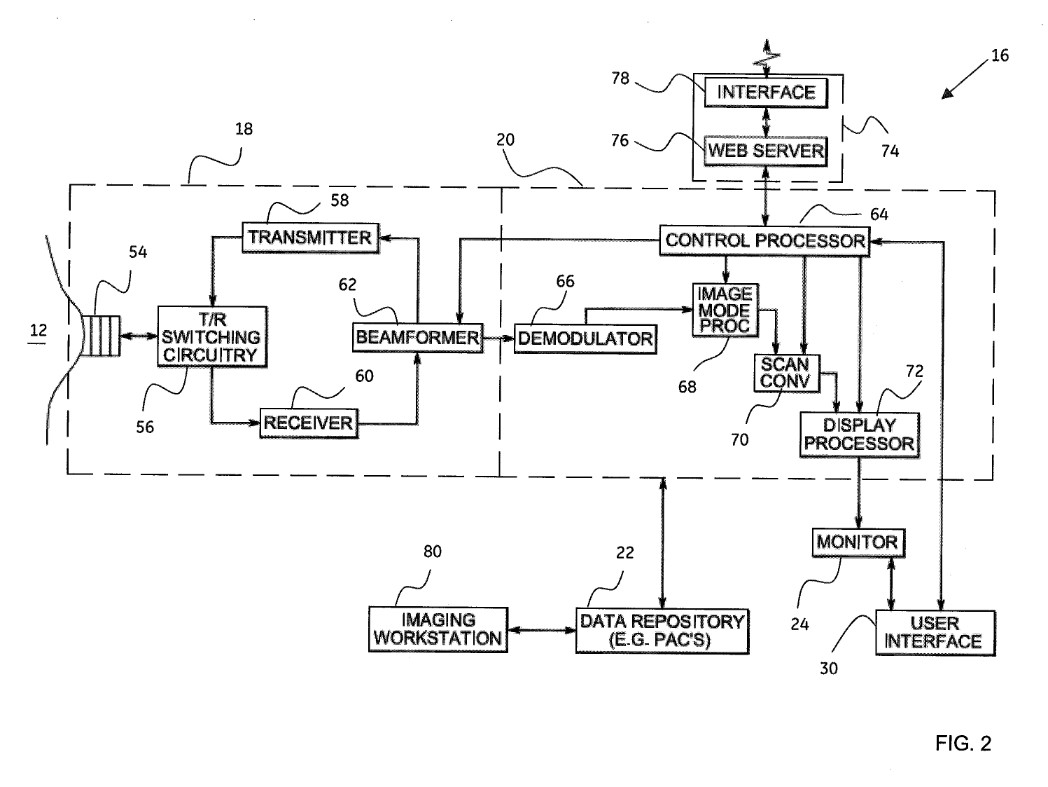

As previously noted, the medical imaging system 16 may include an ultrasound imaging system. FIG. 2 is a block diagram of an embodiment of the medical imaging system 16 of FIG. 1, where the medical imaging system 16 is shown as including an ultrasound imaging system 16. Furthermore, the ultrasound imaging system 16 is shown as including the acquisition subsystem 18 and the processing subsystem 20, as previously described. The acquisition subsystem 18 may include a transducer assembly 54. In addition, the acquisition subsystem 18 includes transmit/receive (T/R) switching circuitry 56, a transmitter 58, a receiver 60, and a beamformer 62. In one embodiment, the transducer assembly 54 may be disposed in the image acquisition device 14 (see FIG. 1). Also, in certain embodiments, the transducer assembly 54 may include a plurality of transducer elements (not shown) arranged in a spaced relationship to form a transducer array, such as a one-dimensional or two-dimensional transducer array, for example. Additionally, the transducer assembly 54 may include an interconnect structure (not shown) configured to facilitate operatively coupling the transducer array to an external device (not shown), such as, but not limited to, a cable assembly or associated electronics. The interconnect structure may be configured to couple the transducer array to the T/R switching circuitry 56.

The processing subsystem 20 includes a control processor 64, a demodulator 66, an imaging mode processor 68, a scan converter 70 and a display processor 72. The display processor 72 is further coupled to a display monitor, such as the single panel display 24 (see FIG. 1), for displaying images. User interface, such as the user interface 30 (see FIG. 1), interacts with the control processor 64 and the display 24. The control processor 64 may also be coupled to a remote connectivity subsystem 74 including a web server 76 and a remote connectivity interface 78. The processing subsystem 20 may be further coupled to the data repository 22 (see FIG. 1) configured to receive ultrasound image data, as previously noted with reference to FIG. 1. The data repository 22 interacts with an imaging workstation 80.

The aforementioned components may be dedicated hardware elements such as circuit boards with digital signal processors or may be software running on a general-purpose computer or processor such as a commercial, off-the-shelf personal computer (PC). The various components may be combined or separated according to various embodiments of the present technique. Thus, those skilled in the art will appreciate that the present ultrasound imaging system 16 is provided by way of example, and the present techniques are in no way limited by the specific system configuration.

In the acquisition subsystem 18, the transducer assembly 54 is acoustically coupled to the patient 12 (see FIG. 1), either by direct contact with the patient 12 or by coupling via an acoustic gel. The transducer assembly 54 is coupled to the transmit/receive (T/R) switching circuitry 56. Also, the T/R switching circuitry 56 is in operative association with an output of the transmitter 58 and an input of the receiver 60. The output of the receiver 60 is an input to the beamformer 62. In addition, the beamformer 62 is further coupled to an input of the transmitter 58 and to an input of the demodulator 66. The beamformer 62 is also operatively coupled to the control processor 64 as shown in FIG. 2.

In the processing subsystem 20, the output of demodulator 66 is in operative association with an input of the imaging mode processor 68. Additionally, the control processor 64 interfaces with the imaging mode processor 68, the scan converter 70 and the display processor 72. An output of the imaging mode processor 68 is coupled to an input of the scan converter 70. Also, an output of the scan converter 70 is operatively coupled to an input of the display processor 72. The output of the display processor 72 is coupled to the display 24.

The ultrasound imaging system 16 transmits ultrasound energy into the patient 12 and receives and processes backscattered ultrasound signals from the patient 12 to create and display an image. To generate a transmitted beam of ultrasound energy, the control processor 64 sends command data to the beamformer 62 to generate transmit parameters to create a beam of a desired shape originating from a certain point at the surface of the transducer assembly 54 at a desired steering angle. The transmit parameters are sent from the beamformer 62 to the transmitter 58. The transmitter 58 uses the transmit parameters to properly encode transmit signals to be sent to the transducer assembly 54 through the T/R switching circuitry 56. The transmit signals are set at certain levels and phases with respect to each other and are provided to individual transducer elements of the transducer assembly 54. The transmit signals excite the transducer elements to emit ultrasound waves with the same phase and level relationships. As a result, a transmitted beam of ultrasound energy is formed in the patient 12 along a scan line when the transducer assembly 54 is acoustically coupled to the patient 12 by using, for example, ultrasound gel. The process is known as electronic scanning.

In one embodiment, the transducer assembly 54 may be a two-way transducer. When ultrasound waves are transmitted into a patient 12, the ultrasound waves are backscattered off the tissue and blood samples within the patient 12. The transducer assembly 54 receives the backscattered waves at different times, depending on the distance into the tissue they return from and the angle with respect to the surface of the transducer assembly 54 at which they return. The transducer elements convert the ultrasound energy from the backscattered waves into electrical signals.

The electrical signals are then routed through the T/R switching circuitry 56 to the receiver 60. The receiver 60 amplifies and digitizes the received signals and provides other functions such as gain compensation. The digitized received signals corresponding to the backscattered waves received by each transducer element at various times preserve the amplitude and phase information of the backscattered waves.

The digitized signals are sent to the beamformer 62. The control processor 64 sends command data to beamformer 62. The beamformer 62 uses the command data to form a receive beam originating from a point on the surface of the transducer assembly 54 at a steering angle typically corresponding to the point and steering angle of the previous ultrasound beam transmitted along a scan line. The beamformer 62 operates on the appropriate received signals by performing time delaying and focusing, according to the instructions of the command data from the control processor 64, to create received beam signals corresponding to sample volumes along a scan line within the patient 12. The phase, amplitude, and timing information of the received signals from the various transducer elements are used to create the received beam signals.

The received beam signals are sent to the processing subsystem 20. The demodulator 66 demodulates the received beam signals to create pairs of I and Q demodulated data values corresponding to sample volumes along the scan line. Demodulation is accomplished by comparing the phase and amplitude of the received beam signals to a reference frequency. The I and Q demodulated data values preserve the phase and amplitude information of the received signals.

The demodulated data is transferred to the imaging mode processor 68. The imaging mode processor 68 uses parameter estimation techniques to generate imaging parameter values from the demodulated data in scan sequence format. The imaging parameters may include parameters corresponding to various possible imaging modes such as B-mode, color velocity mode, spectral Doppler mode, and tissue velocity imaging mode, for example. The imaging parameter values are passed to the scan converter 70. The scan converter 70 processes the parameter data by performing a translation from scan sequence format to display format. The translation includes performing interpolation operations on the parameter data to create display pixel data in the display format.

The scan converted pixel data is sent to the display processor 72 to perform any final spatial or temporal filtering of the scan converted pixel data, to apply grayscale or color to the scan converted pixel data, and to convert the digital pixel data to analog data for display on the display 24. The user interface 30 is coupled to the control processor 64 to allow a user to interface with the ultrasound imaging system 16 based on the data displayed on the display 24.

FIG. 3 illustrates an exemplary portable imaging system 90. In the present example, the portable imaging system 90 may include an ultrasound imaging system, such as the ultrasound imaging system 16 (see FIG. 2). The imaging system 90 may include a hand held imaging system or a hand carried imaging system. Furthermore, the imaging system 90 may include a monolith design. In other words, the imaging system 90 may include a single piece unit. Additionally, the imaging system 90 may be configured to be operationally coupled to a small footprint cart, a pole stand, or a stretcher. Alternatively, the imaging system 90 may be wall mounted.

Further, the imaging system 90 may include a single screen or a single panel display 92. This single panel display 92 may include a single panel display such as the single panel display 24 (see FIG. 1). In accordance with exemplary aspects of the present technique, the single panel display 92 may be configured to serve as both a display area and a user interface area. More particularly, the single panel display 92 may be virtually divided into a first portion 94 and a second portion 98. Here again, the first portion 94 may include a first portion such as the first portion 26 (see FIG. 1), while the second portion 98 may include a second portion such as the second portion 28 (see FIG. 1). The first portion 94 of the single panel display 92 may be configured to facilitate display of an image 96, where the image 96 may be representative of a region of interest in a patient, such as the patient 12 (see FIG. 1), for example.

Furthermore, the second portion 98 of the single panel display 92 may be configured to include a touch-based user interface. The touch-based user interface 98 may be configured to display controls that may be needed to perform a particular scanning task at a time. In other words, the touch-based user interface 98 of the single panel display 92 may be configured to provide a user interface to operating controls that are typically used during an ultrasound scan. Therefore, the controls placed in the touch-based user interface 98 may include controls that are used only for a scanning operation, for example. Moreover, many parameters of the image may be adjusted via use of these touch screen controls in the touch-based user interface 98. For example, the controls in the touch-based user interface 98 may be used to control the way the image appears, the scanning mode that the image is displaying, and the part of the anatomy that is focused upon in the image.

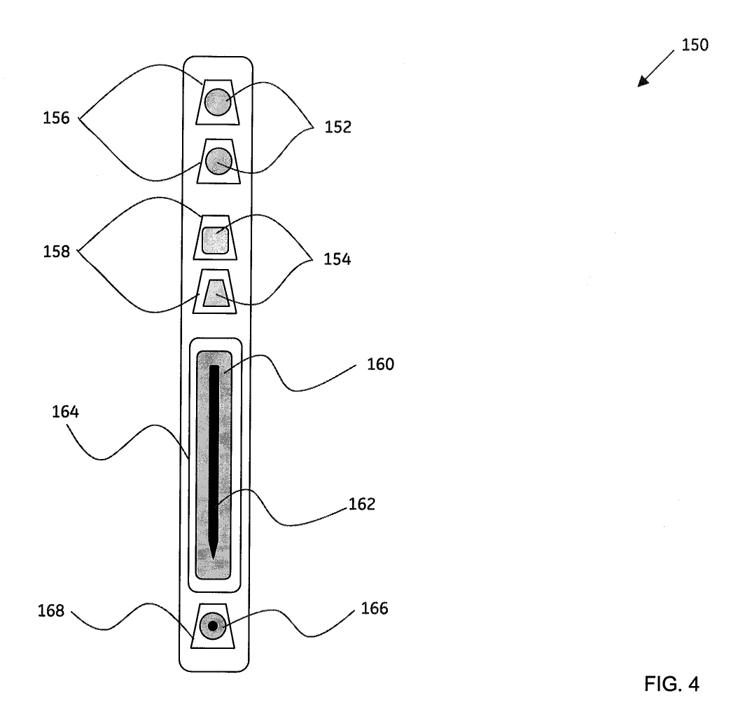

In addition, the imaging system 90 may be configured to dynamically change controls that are displayed in the touch-based user interface 98 based on a mode of operation of the imaging system 90 or a feature being used. By way of example, the controls displayed in the touch-based user interface 98 illustrated in FIG. 3 may be representative of a B-mode of operation of the imaging system 90. In the example illustrated in FIG. 4, the touch-based user interface 98 may be configured to include controls such as Time Gain Compensator (TGC) controls 102, a Focus control 104, a Zoom control 106, a Frequency control 108 and a Depth control 110. In addition, the touch-based user interface 98 may also include an M button 112, a PWD button, a Color button 116 and a Gain button 118. Alternatively, if the imaging system 90 is operating in a color mode, then the imaging system 90 may be configured to display controls specific to the color mode of operation on the touch-based user interface 98. Additionally, the first portion 94 and the second portion 98 of the single panel display 92 may be configured to be virtually separated by a graphical demarcator 100, in certain embodiments. The graphical demarcator 100 may include a graphical demarcator such as the graphical demarcator 29 (see FIG. 1).

In accordance with further aspects of the present technique, the imaging system 90 may also include a controls portion 120. It may be noted that the controls portion 120 may include a user interface such as the user interface 30 (see FIG. 1). Also the controls portion 120 may include one or more buttons, where the buttons may be configured to aid in the imaging of the patient 12 (see FIG. 1). More particularly, the controls portion 120 may be configured to include buttons that may be configured to perform commonly used functions of the imaging system 90. In a presently contemplated configuration, the buttons in the controls portion 120 of the imaging system 90 may include hard buttons. The hard buttons may include hard membrane keys, in certain embodiments.

As noted hereinabove, commonly used functions may be available via hard buttons in the controls portion 120 of the imaging system 90. It may be noted that the hard keys located in controls portion 120 may be representative of keys used to control features outside of a typical scanning operation. Examples of commonly performed functions may include a Print function, a Comment (annotate) function, a Settings function, a Store function, and a Freeze function.

In the example illustrated in FIG. 3, the commonly performed functions may be performed via use of buttons such as a Settings button 122, a Patient button 124, a Comment button 126, a Print button 128, a Store button 130, a Freeze button 132, and a Measure button 134, for example. For example, a clinician may enter patient data using the Patient button 124, while the clinician may take measurements of the image 96 via use of the Measure button 134. Reference numeral 136 may be representative of a mouse pad. Further, reference numeral 138 may be representative of a left click button on the mouse pad 136, where the left click button 138 may be used for setting a cursor, setting a measuring caliper, or clicking on a menu item, for example. Similarly, a right click button on the mouse pad 136 may generally be represented by reference numeral 139, where the right click button 139 may be employed to aid in toggling the cursor on the single panel display 92 between an ON state and an OFF state. In addition, a Power button may generally be represented by reference numeral 140.

By implementing the controls portion 120 as described hereinabove, the hard keys may be separated from the other controls and located in the controls portion 120. By locating these hard keys in the controls portion 120, the hard keys are available at all times, unlike the controls on the touch-based user interface 98 that change depending upon the feature running and/or a mode of operation of the imaging system 90. For example, a Freeze function and a Store function may be applied at any time, independent of a current mode of operation of the imaging system 90, such as a color mode, a Doppler mode, or a B-mode of operation of the imaging system. Also, the commonly used functions, like Freeze, Store, and Depth, may be controlled via use of the membrane covered hard keys. The design of the hard keys allow for tactile feedback, like raised textures and back lighting for good ergonomics and ease of use.

In accordance with further aspects of the present technique, the single panel display 92 and the controls portion 120 may be arranged such that the imaging system 90 includes a seamless form factor of a single unit box. In other words, the single panel display 92 and the controls portion 120 including the hard buttons may have a seamless facade, thereby allowing the console and the screen to be wiped clean with disinfectant and hence prevent places for bacteria to accumulate in hard to clean cracks. Additionally, the seamless design of the facade of the imaging system 90 allows internal components of the imaging system 90 to be protected from fluid splash.

Moreover, in certain embodiments, the imaging system 90 may have a height in a range from about 250 mm to about 300 mm. Also, the imaging system 90 may have a width in a range from about 250 mm to about 300 mm. In addition, the imaging system 90 may have a depth in a range from about 30 mm to about 50 mm. Furthermore, the imaging system 90 may have a weight in a range from about 2 kilograms to about 4 kilograms.

With continuing reference to the imaging system 90, in a presently contemplated configuration, the single panel display 92 may be configured to occupy about 75% of a front face of the imaging system 90. However, as will be appreciated, the single panel display 92 may also be configured to occupy from about 70% to about 90% of the front face of the imaging system 90, in certain other embodiments.

Furthermore, in the example illustrated in FIG. 3, the first portion 94 of the single panel display 92 is shown as occupying about 66% of an area of the single panel display 92, while the touch-based user interface 98 is shown as occupying about 34% of the area of the single panel display 92. However, in accordance with exemplary aspects of the present technique, the area occupied by the first portion 94 and the area occupied by the second portion 98 of the single panel display 92 may be dynamically changed based on a mode of operation of the imaging system 90. In other words, in certain embodiments, it may be desirable to display a relatively larger image on the first portion 94 of the single panel display 92, hence entailing need for increasing the area of the first portion 94, while reducing the area of the second portion 98 of the single panel display 92. Alternatively, in certain other embodiments, it may be desirable to increase an area of the second portion 98 in order to accommodate display of a relatively larger number of controls in the touch-based user interface 98, thereby calling for a reduction in the area of the first portion 94. As previously noted, the first portion 94 and the second portion 98 of the single panel display 92 may be virtually demarcated via the graphical demarcator 100, in certain embodiments.

In accordance with further aspects of the present technique, the imaging system 90 may be configured to automatically adjust a brightness of the single panel display 92 based on current ambient conditions. For example, if the ambient condition includes a substantially bright environment, then the imaging system 90 may be configured to enhance the brightness of the single panel display 92. However, if the ambient condition includes a substantially dark environment, then the imaging system 90 may be configured to accordingly dim the brightness of the single panel display 92. In a presently contemplated configuration, the imaging system 90 may include an ambient light sensor 142, where the ambient light sensor 142 may be configured to aid the imaging system 90 in sensing current ambient conditions and automatically adjusting the brightness of the single panel display 92. Also, in the present example, the ambient light sensor 142 is shown as being located in the controls portion 120. However, the ambient light sensor 142 may also be located else where on the imaging system 90.

As noted hereinabove, the touch-based user interface 98 may be configured to display only a desired set of controls, where the desired set of controls may include buttons that correspond to a particular scanning task being performed. In accordance with exemplary aspects of the present technique, the touch-based user interface 98 may be customized based on a user, such as a clinician, an application, a mode of operation of the imaging system 90, or a combination thereof. In other words, the touch-based user interface 98 may be configured to selectively display controls based on a current mode of operation of the imaging system 90, a user or an application of the imaging system 90.

Furthermore, the imaging system 90 may also include one or more ports, one or more connectors, or both. Referring now to FIG. 4, a diagrammatical illustration of a side view 150 of the imaging system 90 (see FIG. 3) is depicted. Reference numeral 152 may be representative of the one or more ports, while the one or more connectors may generally be represented by reference numeral 154. It may be noted that the one or more ports 152, the one or more connectors 154, or both, may be configured to allow one or more devices to be operationally coupled to the imaging system 90. For example, one or more image acquisition devices, such as, but not limited to, probes, may be coupled to the imaging system 90 via the one or more ports 152 and/or the one or more connectors 154. In addition, the imaging system 90 may also include one or more protective flaps, where the flaps may be configured to cover the ports 152 and/or the connectors 154. In the example depicted in FIG. 4, reference numeral 156 may be representative of the protective flaps configured to cover the ports 152, while the protective flaps configured to cover the connectors 154 may generally be represented by reference numeral 158.

In accordance with aspects of the present technique, the imaging system 90 may be recharged via a freestanding dock, a wall-mounted charging dock, a portable charger adapter, or a combination thereof. Referring again to the embodiment illustrated in FIG. 4, reference numeral 166 may be representative of a charging connector, while a protective flap configured to cover the charging connector may generally be represented by reference numeral 168. The imaging system 90 may also be configured to include a storage area 160 for a touch stylus 162. Reference numeral 164 may generally be representative of a protective flap configured to cover the storage area 160 and/or the stylus 162. It may be noted that these protective flaps 156, 158, 164, 168 may include rubber flaps or silicone flaps, for example.

With returning reference to FIG. 3, a battery (not shown in FIG. 3) in the imaging system 90 may have a life of about one hour. Furthermore, the imaging system 90 may be designed to include a robust unit. For example, the imaging system 90 may be configured to be droppable from a height of about 80 cm, in certain embodiments. Additionally, the imaging system 90 may be configured to boot up in a time of less than about 10 seconds.

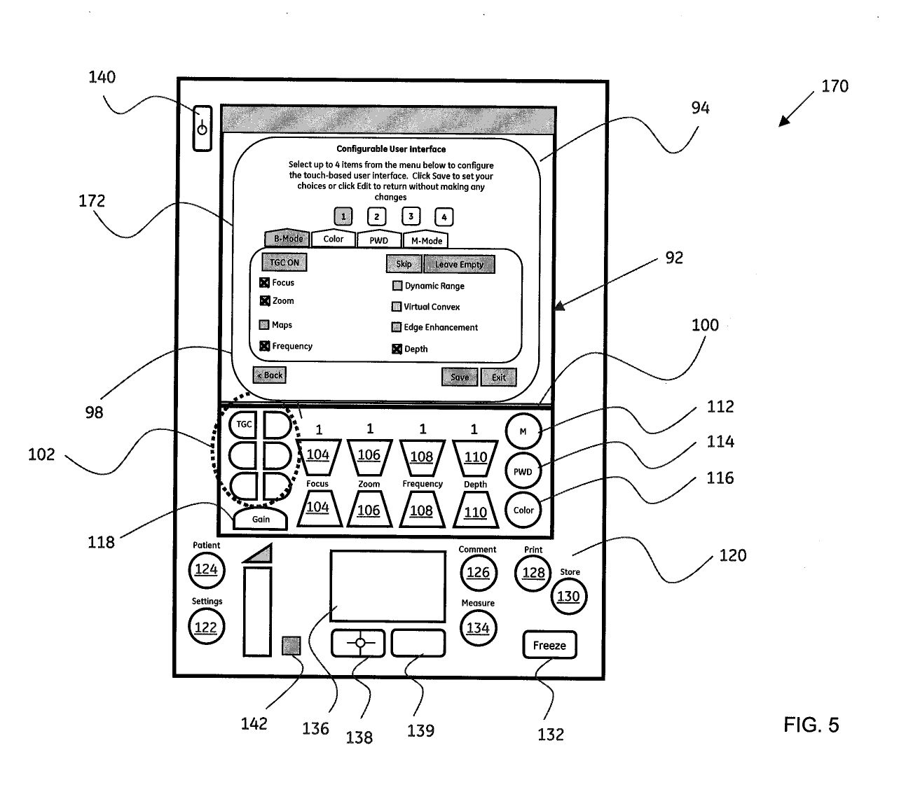

Furthermore, the imaging system 90 may also be configured to allow the user to customize the imaging system 90. More particularly, the user may customize a display of controls on the touch-based user interface 98. In other words, the specifications, parameters, or other utilities of the imaging system 90 may be entered and adjusted via use of the touch-based user interface 98. Additionally, the imaging system 90 may also be configured to allow the user to select user profiles, where the user profiles may include variable settings to suit a corresponding user. The user customization of the imaging system 90 may be better understood with reference to FIG. 5.

Turning now to FIG. 5, a diagrammatic illustration 170 of a method of customizing the imaging system 90 (see FIG. 3) using the touch-based user interface 98 (see FIG. 3) is depicted. In the example of FIG. 5, the user may customize the imaging system 90 and more particularly the touch-based user interface 98 to include user selected controls. In a presently contemplated configuration, the user may customize the touch-based user interface 98 via use of the Settings button 122. Once the user selects the Settings button 122, the imaging system 90 may be configured to display a dialog box 172. The user may then customize the imaging system 90 by selecting one or more controls displayed on the dialog box 172, thereby updating the display of controls on the touch-based user interface 98.

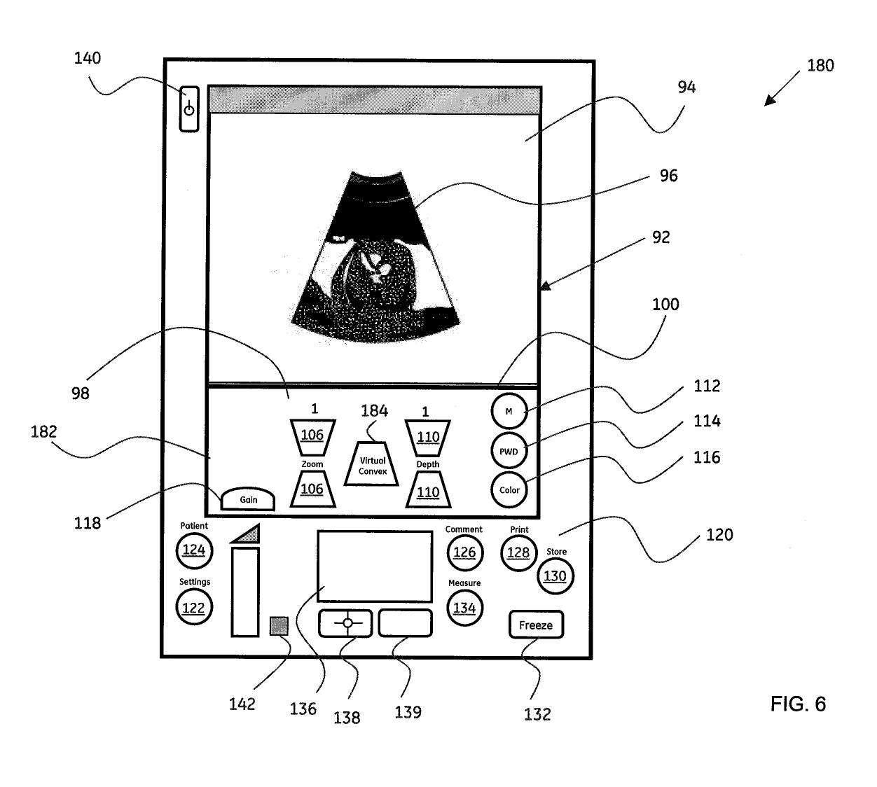

An example 180 of a user defined control panel 182 is illustrated in FIG. 6. It may be noted that user defined control panel 182 may be representative of the touch-based user interface 98 (see FIG. 3) that is modified based on the user selection illustrated in FIG. 5. The user defined control panel 182 may be configured to include the Gain button 118, the Zoom control 106, the Depth control 110, the M button 112, the PWD button 114, and the Color button 116. In addition, the user defined control panel 162 may also be configured to include a Virtual Convex button 184. It may be noted that the TGC controls 102 (see FIG. 3), the Focus control 104 (see FIG. 3), and the Frequency control 108 (see FIG. 3) have been deleted from the user defined control panel 182 and replaced by other buttons, such as the Virtual Convex button 184. Additionally, locations of the Zoom control 106 and the Depth control 110 have been rearranged.

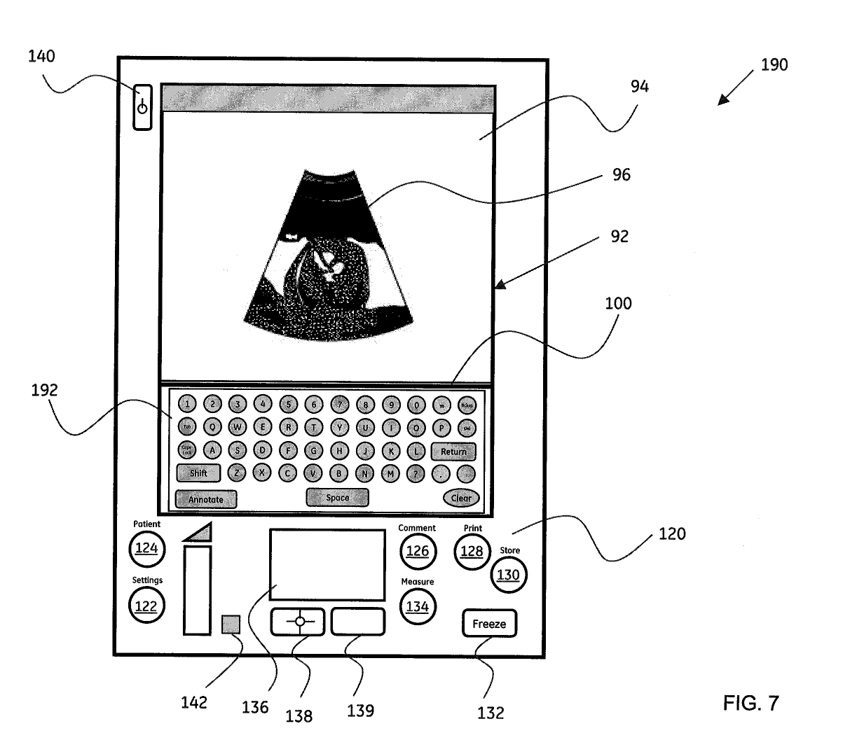

Moreover, it may be desirable for the user to annotate an image, such as the image 96 (see FIG. 3). In accordance with aspects of the present technique, the imaging system 90 may be configured to allow the clinician to annotate an image displayed on the first portion 94 of the single panel display 92. FIG. 7 is a diagrammatic illustration 190 of a method of annotating an image via use of the touch-based user interface 98. Accordingly, the imaging system 90 may be configured to display a touch panel keyboard 192 on the second portion 98 of the single panel display 92 (see FIG. 3). The clinician may then annotate the image 96 using this touch-panel keyboard 192. In one embodiment, the touch-panel keyboard 192 may be displayed on the touch-based user interface 98 via selection of the Comment button 126.

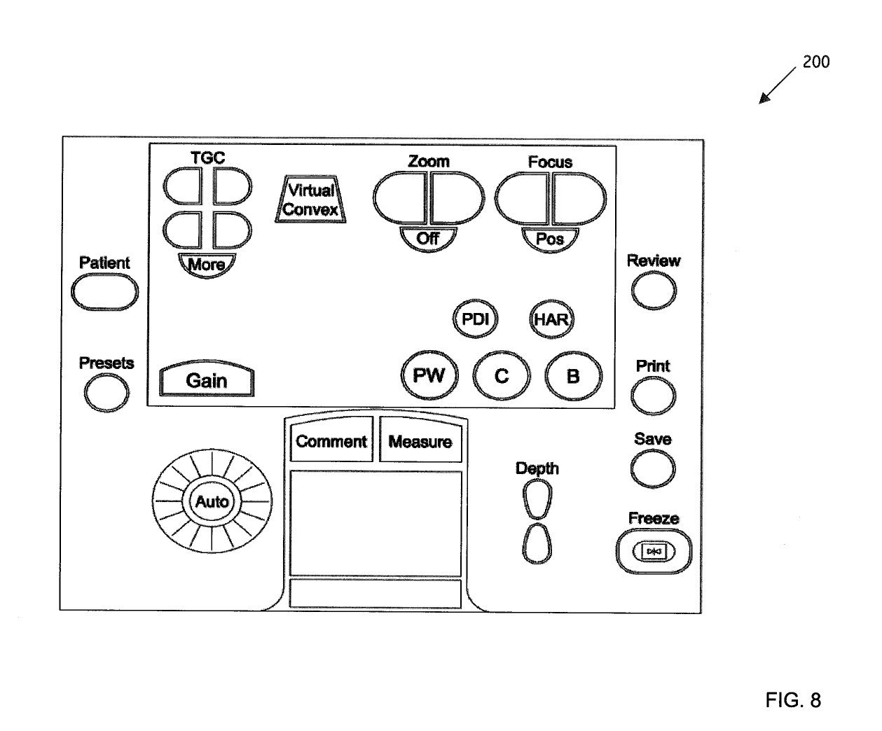

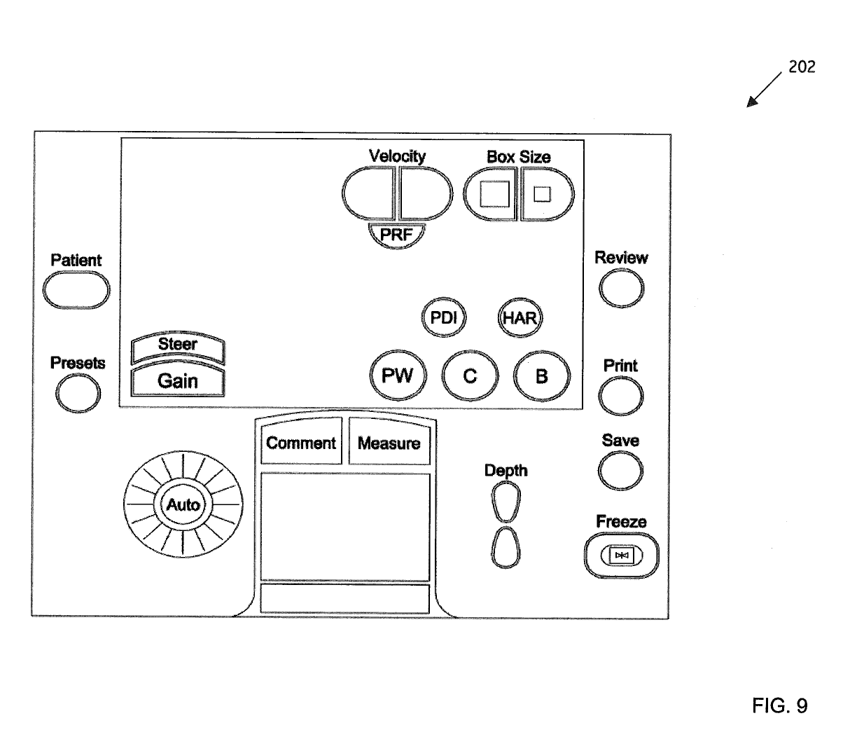

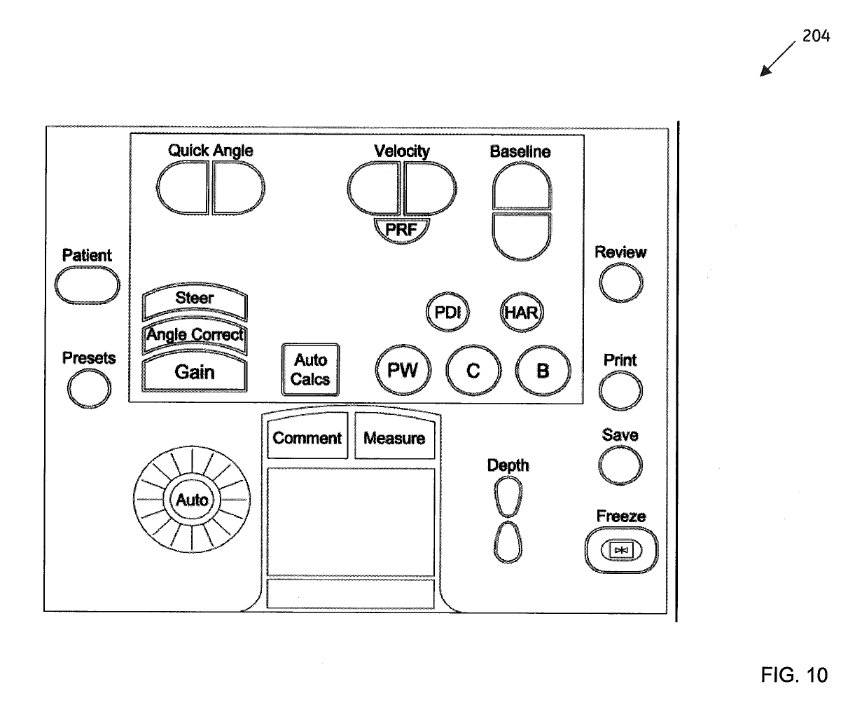

In accordance with further aspects of the present technique, the imaging system 90 and more particularly the touch-based user interface 98 may be configured to selectively display controls based on a current mode of operation of the imaging system 90. As will be appreciated, the imaging system 90 may be operated in a B-mode, a Color mode, or a Doppler mode. FIG. 8 illustrates one embodiment 200 of the touch-based user interface 98 (see FIG. 3) showing controls corresponding to a B-mode of operation of the imaging system 90 (see FIG. 3). Referring now to FIG. 9, an embodiment 202 of the touch-based user interface 98 (see FIG. 3) showing controls corresponding to a Color mode of operation of the imaging system 90 (see FIG. 3) is illustrated. Further, FIG. 10 illustrates one embodiment 204 of the touch-based user interface 98 (see FIG. 3) showing controls corresponding to a Doppler mode of operation of the imaging system 90 (see FIG. 3).

By implementing the imaging system as described hereinabove, a portable, simple to use imaging system may be produced, where the imaging system has a seamless facade. This design advantageously allows the imaging system to be easily cleaned and hence allow use of the imaging systems in sterile environments.

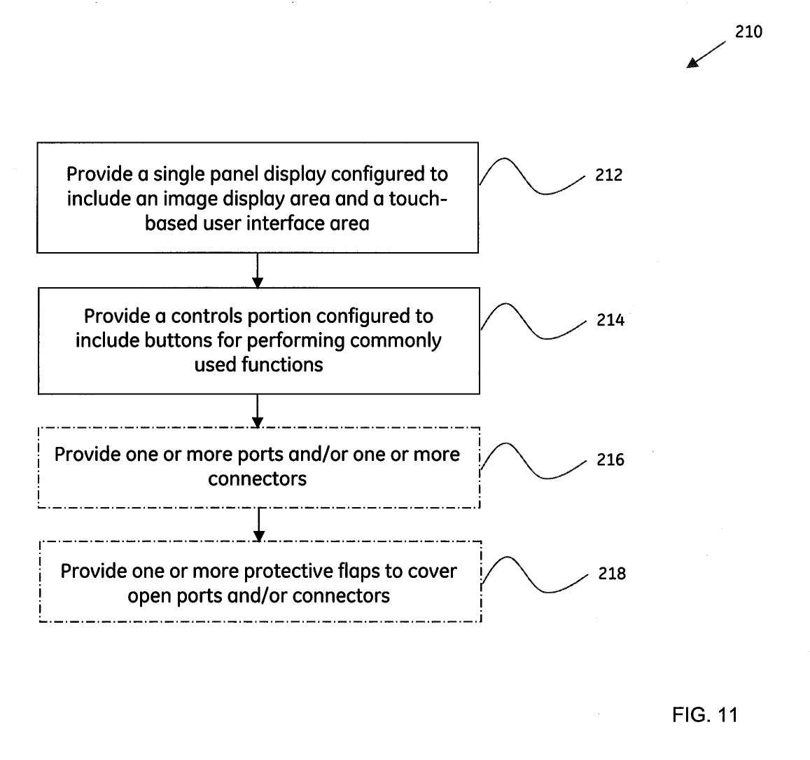

In accordance with further aspects of the present technique, a method of making the exemplary imaging system 90 of FIG. 3 is presented. Turning now to FIG. 11, a flow chart 210 illustrating the exemplary method of making the portable imaging system 90 is depicted. The method starts at step 212, where a single panel display, such as the single panel display 92 (see FIG. 3) may be provided. This single panel display may be configured to include a first portion and a second portion, where the first portion may be configured to display an image, while the second portion may be configured as a touch-based user interface. As previously noted, an image representative of one or more regions of interest of the patient 12 (see FIG. 1) may be displayed on the image area of the single panel display. In addition, the touch-based user interface of the single panel display may be configured to display one or more controls to aid the clinician in performing imaging tasks.

Further, at step 214, a controls portion may be provided. The controls portion may be configured to include one or more buttons, where the buttons may be configured to aid in performing commonly used functions, such as a Print function, a Store function, a Freeze function, or the like. Also, these buttons on the controls portion may be configured to include hard membrane keys.

Additionally, at step 216, one or more ports may be provided, where the one or more ports may be configured to facilitate operatively coupling components such as probes to the imaging system. Moreover, at step 218, one or more protective flaps may be provided, where the flaps may be configured to cover any open ports and/or connectors, thereby protecting the ports and/or connectors from fluid splash.

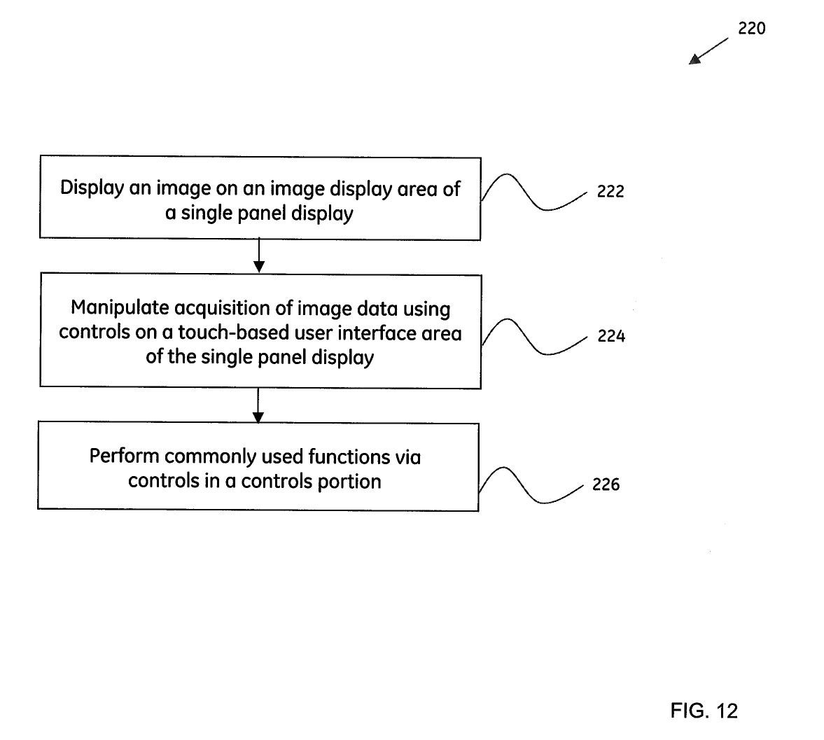

In accordance with further aspects of the present technique, a method of imaging using the exemplary imaging system 90 of FIG. 3 is presented. Turning now to FIG. 12, a flow chart 220 illustrating the exemplary method of imaging using the portable imaging system 90 is depicted. As previously described, the imaging system 90 may include a single panel display 92 (see FIG. 3), where the single panel display 92 may be virtually divided into the image display portion 94 (see FIG. 3) and the touch-based user interface 98 (see FIG. 3). In addition, the imaging system 90 may also include the controls portion 120 (see FIG. 3). The method starts at step 222, where an image representative of one or more regions of interest in the patient 12 (see FIG. 1) may be displayed on a first portion of the single panel display of the imaging system. Further, at step 224, the acquisition of image data and/or the displayed image may be manipulated via use of controls on the touch-based user interface 98. Moreover, commonly used functions, such as, but not limited to, Print, Freeze, or Store, may be performed via use of buttons on the controls portion of the imaging system, as depicted in step 226.

Additionally, the touch-based user interface may be customized based on a user, an application, a mode of operation, or a combination thereof. As previously noted, customization of the touch-based user interface may include selectively displaying controls based on a current mode of operation of the imaging system. Furthermore, an area of the image display portion and an area of the touch-based user interface may be dynamically modified based on the current mode of operation of the imaging system. Controls available on the controls portion of the imaging system may also be used for perform commonly used functions, such as Print, Freeze, Store, or the like. Moreover, the imaging system may also be recharged via a free standing charging dock, a wall mounted charging dock, a portable charger adaptor, or a combination thereof. Also, a brightness of the single panel display may be automatically adjusted based on ambient lighting conditions.

The exemplary portable imaging system and the method for imaging using the exemplary portable imaging system described hereinabove dramatically enhance clinical workflow as the substantially small size of the imaging system allows the imaging system to fit into more tight, crowded rooms, like the operating room or an emergency crash room. The imaging system may also be attached to an intra-venous (IV) pole already in the room or to a small stand. Further, the imaging system may be hand carried from room to room as needed. Moreover, the imaging system may be placed into a wall mounted charging dock that is out of the way but readily available. Also, the imaging system may be fit into cramped areas like ambulances and helicopters.

In addition, the exemplary touch-based user interface portion of the single panel display allows for more simple operation of the scanning controls. Furthermore, the controls displayed on the touch-based user interface may be customized based on the user, the application, or the mode of operation, thereby simplifying the imaging process. Moreover, the seamless design of the console of the imaging system allows users to quickly wipe the imaging system with disinfectant, thereby saving time and allowing the imaging system to be used in exacting environments, such as sterile operating rooms.

The above-description of the embodiments of the portable imaging system and the method for imaging using the portable imaging system have the technical effect of enhancing clinical workflow as the monolith design of the exemplary portable imaging system is small and may be hand carried and/or hand held. Additionally, the small, one piece unit has a single panel display with a portion dedicated to displaying an image and a portion dedicated to touch-panel controls to operate the system. The touch-panel controls display only the buttons needed to perform a particular scanning task at a time. Furthermore, a few commonly used buttons are still conventional hard buttons on the console. Additionally, the single screen scanning/operating controls, membrane covered hard keys, and seamless form factor of the single unit box allow the console and screen to wiped clean with disinfectant and prevent places for bacteria to accumulate in hard to clean cracks. Also, the internal components of the system may be protected from fluid splash.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A portable imaging system, comprising:
a single panel display, wherein the single panel display comprises a first portion configured to display an image and a second portion configured as a touch-based user interface.

2. The system of claim 1, further comprising a controls portion, wherein the controls portion comprises one or more buttons configured to aid in performing commonly used functions.

3. The system of claim 2, wherein the single panel display and the controls portion comprise a seamless form factor of a single unit box.

4. The system of claim 1, wherein the system comprises a hand holdable or a hand carryable imaging system.

5. The system of claim 4, wherein the system comprises an ultrasound imaging system.

6. The system of claim 1, wherein the touch-based user interface comprises controls to operate the imaging system.

7. The system of claim 1, wherein the touch-based user interface is configured to be customized based on a user, an application, a mode of operation, or a combination thereof.

8. The system of claim 7, wherein the touch-based user interface is configured to selectively display controls based on the customization.

9. The system of claim 8, wherein an area of the first portion and an area of the second portion of the single panel display is dynamically changed based on the customization.

10. The system of claim 1, further comprising one or more ports, one or more connectors, or both, wherein the one or more ports, the one or more connectors, or both, are configured to facilitate operatively coupling one or more probes to the system.

11. The system of claim 10, further comprising one or more protective flaps configured to cover any open ports or connectors.

12. The system of claim 1, wherein the system is configured to be recharged via a free standing charging dock, a wall mounted charging dock, a portable charger adaptor, or a combination thereof.

13. The system of claim 1, wherein the system is configured to automatically adjust a brightness of the single panel display based on ambient lighting conditions.

14. The system of claim 1, further comprising storage for a touch stylus.

15. A method of making a portable imaging system, the method comprising:
providing a single panel display, wherein the single panel display comprises a first portion configured to display an image and a second portion configured as a touch-based user interface.

16. The method of claim 15, further comprising providing a controls portion, wherein the controls portion comprises one or more buttons configured to aid in performing commonly used functions.

17. The method of claim 16, further comprising providing one or more ports, one or more connectors, or both, wherein the one or more ports, the one or more connectors, or both, are configured to facilitate operatively coupling one or more probes to the system.

18. The method of claim 17, further comprising providing one or more protective flaps configured to cover any open ports or connectors.

19. A method of imaging using a portable imaging system, wherein the portable imaging system comprises:
a single panel display, wherein the single panel display comprises a first portion configured to display an image and a second portion configured as a touch-based user interface;
a controls portion, wherein the controls portion comprises one or more buttons configured to aid in performing commonly used functions;
the method comprising:
displaying an image on the first portion of the single panel display; and
manipulating the image using controls on the touch-based user interface.

20. The method of claim 19, further comprising customizing the touch-based user interface based on a user, an application, a mode of operation, or a combination thereof.

21. The method of claim 20, wherein customizing the touch-based user interface comprises selectively displaying controls based on the customization.

22. The method of claim 21, further comprising dynamically modifying an area of the first portion and an area of the second portion of the single panel display based on the customization.

23. The method of claim 19, further aiding in performing commonly used functions via controls in the controls portion of the system.

24. The method of claim 19, further comprising automatically adjusting a brightness of the single panel display based on ambient lighting conditions.

25. A computer readable medium comprising one or more tangible media, wherein the one or more tangible media comprise:
code adapted to display an image on a first portion of a single panel display; and
code adapted to manipulate the image using a touch-based user interface on a second portion of the single panel display.
